(19) United States
(12) Patent Application Publication
(10) Pub. No.: US 2024/0408287 A1
(43) Pub. Date: Dec. 12, 2024

(54) BREAST MILK COLLECTION DEVICE

(71) Applicant: Acute Ideas Co., Ltd., Taipei City (TW)

(72) Inventors: Yu-Yun Dong, Tainan City (TW); Chao-Shun Ko, Taipei (TW)

(21) Appl. No.: 18/467,824

(22) Filed: Sep. 15, 2023

(30) Foreign Application Priority Data
Jun. 9, 2023 (CN) 202321484716.4

(51) Int. Cl. A61M 1/06

(52) U.S. Cl. CPC A61M 1/064; A61M 1/062; A61M 1/069

(57) ABSTRACT

The present invention introduces a breast milk collection device with a first shell, a second shell, and a membrane. The first shell has a milk suction passage and a milk accumulation chamber with an opening. The second shell is connected to the first shell and has a fixing seat. The membrane includes a negative pressure diaphragm and an annular one-way valve diaphragm connected to its outer edge. The membrane is adjustable, the negative pressure diaphragm tightly seals the milk suction passage, and the one-way valve diaphragm can block the opening. A negative pressure cavity forms between the negative pressure diaphragm and the second shell. The breast milk collection device features a durable, simple, and easily assembled one-way valve for efficient breast milk collection.

### CROSS REFERENCE TO RELATED APPLICATIONS

This Non-provisional application claims priority under 35 U.S.C. § 119(a) on Patent Application No. 202321484716.4 filed in People's Republic of China on Jun. 9, 2023, the entire contents of which are hereby incorporated by reference.

### BACKGROUND

### 1. Technical Field

The present invention relates to the technical field of maternal and infant products, in particular to a breast milk collection device.

### 2. Description of Related Art

The breast milk collection device means a tool used to express breast milk that has accumulated in the mammary glands. The breast milk collection device has the advantages of being convenient for professional women to breastfeed, solving the sucking difficulty by baby, helping to dredge the mammary gland, and determining the amount of milk, so it is widely used in breastfeeding for modern women.

Refer to the patent literature with the publication number CN101553264B, which is a conventional breast milk collection device. A valve in the conventional breast milk collection device is set as a structure adaptable for the shape and size of an opening of the breast milk collection device. In long-term use, the valve is prone to deformation and cannot provide a good blocking effect on the opening, which in turn affects the failure of the breast milk collection device to perform normal negative pressure breastfeeding, and the blocking member (that is, the valve) needs to be replaced to use normally. Therefore, the consumption cost is increased, and the user experience is reduced.

Therefore, it is necessary to provide a technical solution to solve the above problems.

### SUMMARY OF THE INVENTION

In view of the foregoing, the object of the present invention is to provide a breast milk collection device to solve the problems that the existing breast milk collection device has a complex structure, inconvenient assembly, and the service life of the valve is not long and needs to be replaced frequently, which increases the consumption cost of the user.

To achieve the above, the breast milk collection device includes a first shell, a second shell, and a membrane.

A middle portion of the first shell is provided with a milk suction passage and a milk accumulation chamber outwardly formed by a side wall of the milk suction passage, and a side end of the milk accumulation chamber is provided with an opening through the first shell. The second shell is hermetically connected to the first shell, and a fixing seat is disposed to the second shell. The membrane has a negative pressure diaphragm and an annular one-way valve diaphragm. The annular one-way valve diaphragm is connected to an outer edge of the negative pressure diaphragm. The negative pressure diaphragm is installed on the fixing seat with an adjustable angle and closely contacts the first shell to block the milk suction passage. A negative pressure cavity is formed between the negative pressure diaphragm and the second shell. The opening is blocked by contact with the first shell by the one-way valve diaphragm. A milk storage cavity is formed by the cooperation of the first shell and the second shell on an outside of the fixing seat. A milk discharging hole and a negative pressure hole are disposed on the second shell, wherein the milk discharging hole is connected to the milk storage cavity, and the negative pressure hole is connected to the negative pressure cavity.

In one embodiment, the second shell has a stopper protrusion on both sides of the opening, and the stopper protrusions are in contact with the one-way valve diaphragm.

In one embodiment, there are a plurality of reinforcing protrusions disposed on the one-way valve diaphragm.

In one embodiment, the first shell has a cover part, a tube body, and a membrane seat, which are integrally formed. The cover part is trumpet-shaped. The milk suction passage is disposed from a middle portion of the cover part and runs through the tube body and the membrane seat in turn. The milk accumulation chamber and the opening are disposed on the membrane seat. The negative pressure diaphragm is in close contact with the membrane seat.

In one embodiment, the breast milk collection device further includes an annular groove, an annular block, and a bending portion. The annular groove is disposed inside the fixing seat. The annular block is disposed on the membrane seat, and the annular block is surrounded outside the milk suction passage. The bending portion is disposed on the negative pressure diaphragm, and two sides of the bending portion are in close contact with a wall of the annular block and a wall of the annular groove respectively.

In one embodiment, a convex ring is disposed on the bending portion that the convex ring is in close contact with an inner wall of the annular groove.

In one embodiment, a connecting part is disposed on the second shell, wherein an end of the connecting part is provided with a connecting groove communicated with the milk discharging hole, and a wall surface of the connecting groove is provided with an internal thread.

In one embodiment, the breast milk collection device further includes an annular sealing groove and an annular sealing ring. The annular sealing groove is formed on the first shell. The annular sealing ring is formed by protrusions on the second shell, wherein the sealing ring is located inside the sealing groove.

In one embodiment, an included angle formed between an axis of the negative pressure diaphragm and a normal line of the one-way valve diaphragm is −5° to 5°.

In one embodiment, an included angle formed between an axis of the membrane seat and a normal line of the plane where the opening is located is −5° to 5°.

The technical effects of the breast milk collection device of the present invention include the following:

In the present application, the one-way valve diaphragm connected to the negative pressure diaphragm is designed in a ring shape through reasonable structural settings, so that the one-way valve diaphragm has no directionality when used. When the one-way valve diaphragm is partially deformed due to long-term use resulting it can no longer block the opening, the angle and position of the one-way valve diaphragm can be adjusted by rotating the membrane, and the one-way valve diaphragm can use the rest of the diaphragm to continue to cover and block the opening so that expression can proceed normally. By adopting the design of the present application, the service life of the membrane can be extended, and the consumption cost of the user can be reduced, and the membrane has a simple structure and is convenient to assemble.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

FIG. 1 is a schematic diagram showing the structure of a breast milk collection device according to the first embodiment of the invention.

FIG. 2 is an exploded view showing the structure of the breast milk collection device according to the first embodiment of the invention.

FIG. 3 is an enlarged schematic diagram of the part A in FIG. 1.

FIG. 4 is an enlarged schematic diagram of the part B in FIG. 1.

FIG. 5 is a schematic diagram showing the structure of the second shell of the breast milk collection device according to the first embodiment of the invention.

FIG. 6 is a schematic cross-sectional view of the membrane of the breast milk collection device according to the first embodiment of the invention.

FIG. 7 is a schematic cross-sectional view of the first shell of the breast milk collection device according to the first embodiment of the invention.

FIG. 8 is a schematic diagram showing the structure of a breast milk collection device according to the second embodiment of the invention.

FIG. 9 is an enlarged schematic diagram of the part E in FIG. 8.

FIG. 10 is a perspective view of FIG. 8 from another perspective.

### DETAILED DESCRIPTION

In order to provide a clearer understanding of the objectives, technical solutions, and advantages of the present invention, the following detailed description is provided in conjunction with the drawings and embodiments. It should be understood that the specific embodiments described here are intended to illustrate the present invention and are not intended to limit the scope of the invention.

It should be noted that when an element is referred to as “located on” or “disposed on” another element, it may be directly attached to the other element or may exist with an intermediate element. when an element is referred to as “connected to” or “communicated with” another element, it may be directly connected to or communicated with another element or there may also be an intervening element present together.

In addition, the terms “first” and “second” are used solely for descriptive purposes and should not be construed as indicating or implying relative importance or implying the quantity of the indicated technical features. Therefore, features labeled as “first” or “second” may expressly or implicitly include one or more of such features. In the context of the present invention, the term “a plurality of” means two or more, unless otherwise explicitly specified.

In the description of the embodiments of the present invention, it should be understood that terms such as “upper”, “lower”, “front”, “rear”, “left”, “right”, “top”, “bottom”, “inner” and “outer” and the like indicating orientations or positional relationships are based on the orientations or positional relationships shown in the drawings. These terms are used solely for the purpose of facilitating the description and simplifying the explanation of the embodiments of the present invention. They do not imply or suggest that the devices or components referred to must have specific orientations, be constructed and operated in specific orientations, and therefore should not be construed as limiting the scope of the present invention.

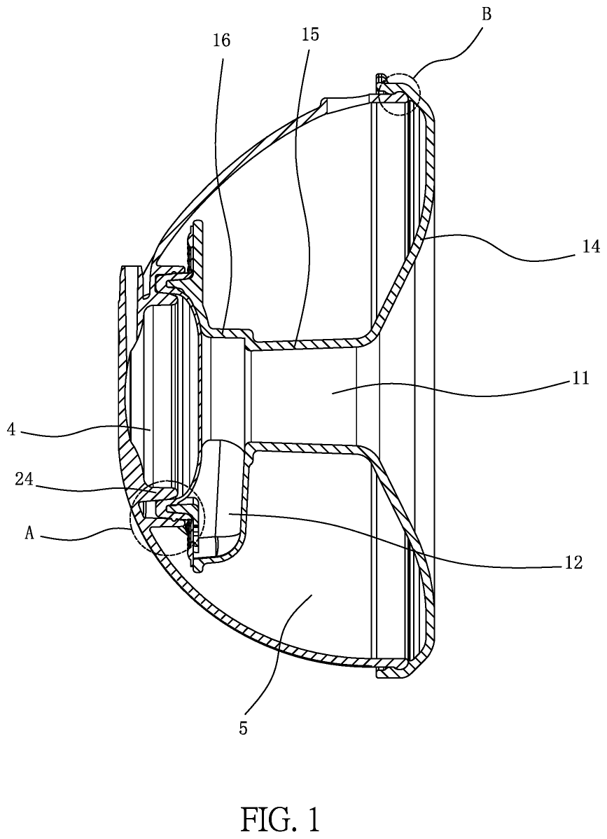

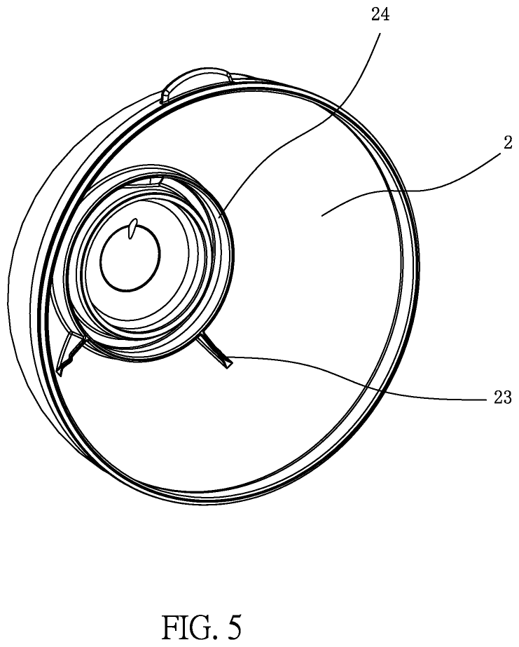

In order to illustrate the technical solution of the present invention more clearly, a preferred embodiment is provided below. Referring to FIG. 1 to FIG. 8, a breast milk collection device includes a first shell 1, a second shell 2, and a membrane 3.

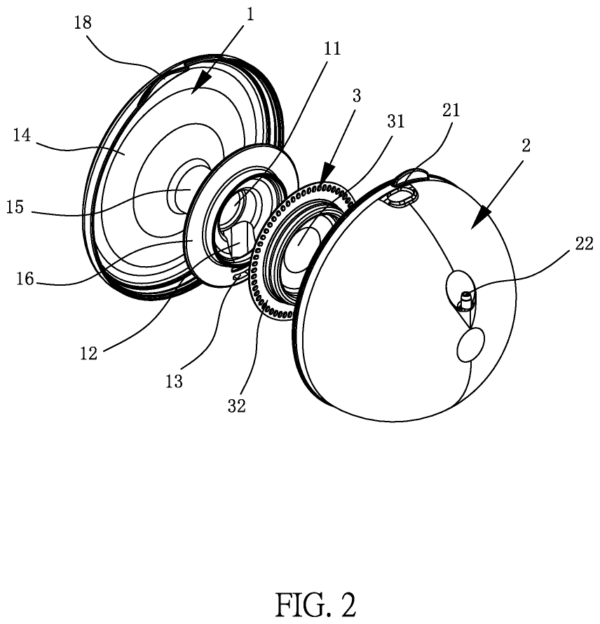

Refer to FIG. 1 and FIG. 2, a middle portion of the first shell 1 is provided with a milk suction passage 11 and a milk accumulation chamber 12 outwardly formed by a passage wall of the milk suction passage 11, and a side end of the milk accumulation chamber 12 is provided with an opening 13 through the first shell 1. It should be noted that the term “outwardly formed” here may refer to extending outward from a passage opening; thus, the opening 13 of the milk accumulation chamber 12 and the passage opening of the milk suction passage 11 are not on the same plane. The second shell 2 is hermetically connected to the first shell 1, and a fixing seat 24 is disposed to the second shell 2. The membrane 3 has a negative pressure diaphragm 31 and an annular one-way valve diaphragm 32. The annular one-way valve diaphragm 32 is connected to an outer edge of the negative pressure diaphragm 31. The negative pressure diaphragm 31 is installed on the fixing seat 24 with an adjustable angle and closely contacts the first shell 1 to block the milk suction passage 11. A negative pressure cavity 4 is formed between the negative pressure diaphragm 31 and the second shell 2. The opening 13 is blocked by the one-way valve diaphragm 32 through contact with the first shell 1. In detail, the negative pressure diaphragm 31 seals the space formed by the passage opening, the milk accumulation chamber 12, and the first shell 1, additionally, the one-way valve diaphragm 32 is contacted to the first shell 1 seal the opening 13, furthermore, the opening 13 and the one-way valve diaphragm 32 constitute a one-way valve. At the outside of the fixing seat 24, the cooperation of the first shell 1 and the second shell 2 forms a milk storage cavity 5. A milk discharging hole 21 and a negative pressure hole 22 are disposed on the second shell 2, wherein the milk discharging hole 21 is connected to the milk storage cavity 5, and the negative pressure hole 22 is connected to the negative pressure cavity 4.

In the invention, the one-way valve diaphragm 32 connected to the negative pressure diaphragm 31 is designed in a ring shape through thoughtful structural arrangements. This ensures that the one-way valve diaphragm 32 does not have a specific directionality when used. In the event that the one-way valve diaphragm 32 undergoes partial deformation after prolonged use, leading to an inability to block the opening 13 effectively, the angle and position of the one-way valve diaphragm 32 can be adjusted by rotating the membrane 3. This allows the remaining portion of the one-way valve diaphragm 32 to continue covering and blocking the opening 13 so that expression can proceed normally. By adopting the design of the present application, the service life of the membrane 3 can be extended, and the consumption cost of the user can be reduced, and the membrane 3 has a simple structure and is convenient to assemble.

Further, the working process of the breast milk collection device includes the following processes. The first shell 1 is used to touch or support the breast, with the nipple positioned in the milk suction passage 11. During breastfeeding, the air in the negative pressure cavity 4 is extracted through a negative pressure device, so that the negative pressure diaphragm 31 is deformed, and the volume of the milk suction passage 11 is increased to generate negative pressure. Consequently, the negative pressure causes the breast and the one-way valve diaphragm 32 to be drawn, leading to milk extraction from the nipple, which then enters the milk accumulation chamber 12 for temporary storage. When the pressure in the milk suction passage 11 returns to approximately atmospheric pressure, the one-way valve diaphragm 32 loses its blocking effect on milk, allowing the milk in the milk accumulation chamber 12 to flow into the milk storage cavity 5 through the opening 13. Subsequently, the negative pressure device extracts air from the negative pressure cavity 4 again, repeating the cycle to collect a certain amount of milk.

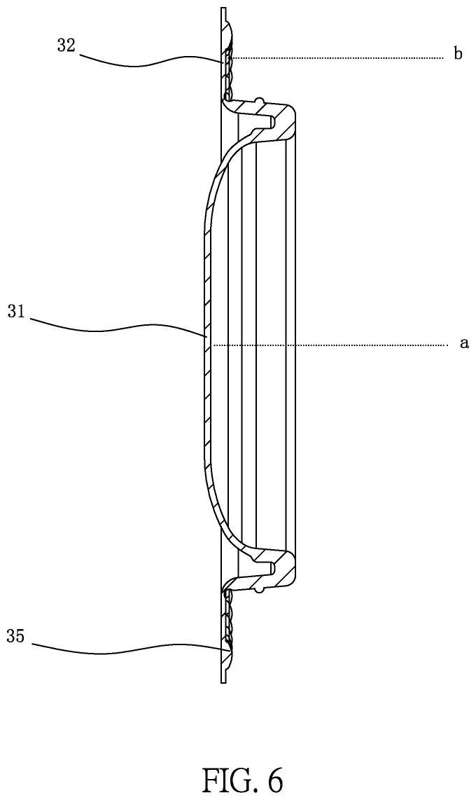

Refer to FIG. 6, in the embodiment, the angle formed between the axis (a) of the negative pressure diaphragm 31 and the normal line (b) of the one-way valve diaphragm 32 is set to be within the range of −5° to 5°. When the aforementioned angle exceeds the range value, it may lead to an unsmooth discharge of milk, thereby affecting the smooth progress of normal milk pumping work.

Refer to FIGS. 2, 5, and 6, in the embodiment, the second shell 2 is provided with a stopper protrusion 23 on both sides of the opening 13. These stopper protrusions 23 are in contact with the one-way valve diaphragm 32 and restrict the movement of the one-way valve diaphragm 32. Specifically, during the discharge of milk from the milk accumulation chamber 12, only a portion of the one-way valve diaphragm 32 between the two stopper protrusions 23 is allowed to move. This design ensures that the remaining portion of the one-way valve diaphragm 32 remains inactive and serves as a reliable backup. Additionally, the stopper protrusions 23 effectively limit the influence of external factors, such as gravity or assembly irregularities, on the action of the one-way valve diaphragm 32.

Refer to FIGS. 1, 2, and 3, in the embodiment, an annular groove 33 is provided at the junction of the negative pressure diaphragm 31 and the one-way valve diaphragm 32. Adopting the above design can make the wall thickness at the junction between the negative pressure diaphragm 31 and the one-way valve diaphragm 32 thinner than the wall thickness of the negative pressure diaphragm 31 and the one-way valve diaphragm 32. This ensures that the one-way valve diaphragm 32 can be assembled flat on the membrane seat 16.

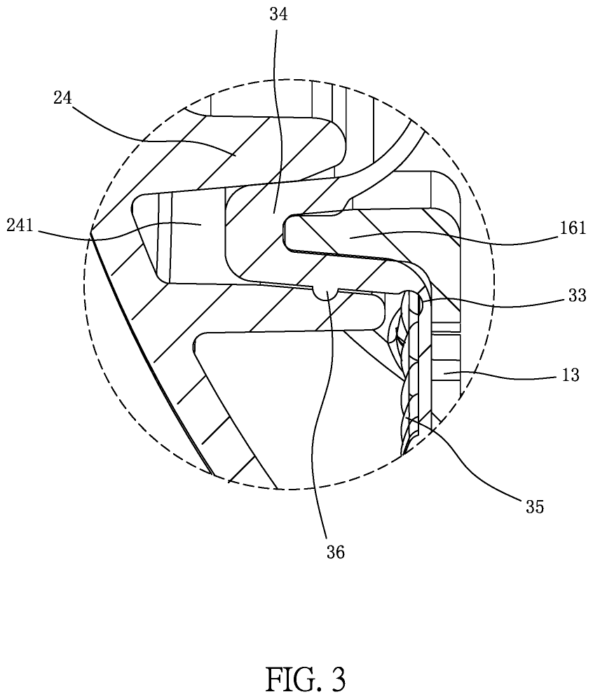

Refer to FIGS. 3 and 6, in the embodiment, a plurality of reinforcing protrusions 35 are provided on the one-way valve diaphragm 32. When the one-way valve diaphragm 32 is subjected to negative pressure suction, it may sag and cause air leakage. The addition of the reinforcing protrusions 35 enhances the strength of the one-way valve diaphragm 32, preventing air leakage while maintaining its flexural softness.

Refer to FIGS. 1, 2, and 7, in the embodiment, the first shell 1 is composed of a cover part 14, a tube body 15, and a membrane seat 16, all integrally formed. The cover part 14 has a trumpet-shaped design. The milk suction passage 11 originates from the middle of the cover part 14 and runs through the tube body 15 and the membrane seat 16 sequentially. Both the milk accumulation chamber 12 and the opening 13 are situated on the membrane seat 16. The negative pressure diaphragm 31 is closely contacted to the membrane seat 16. The adoption of a trumpet-shaped structure for the cover part 14 allows for a better fit with the breast, ensuring a more stable breastfeeding process.

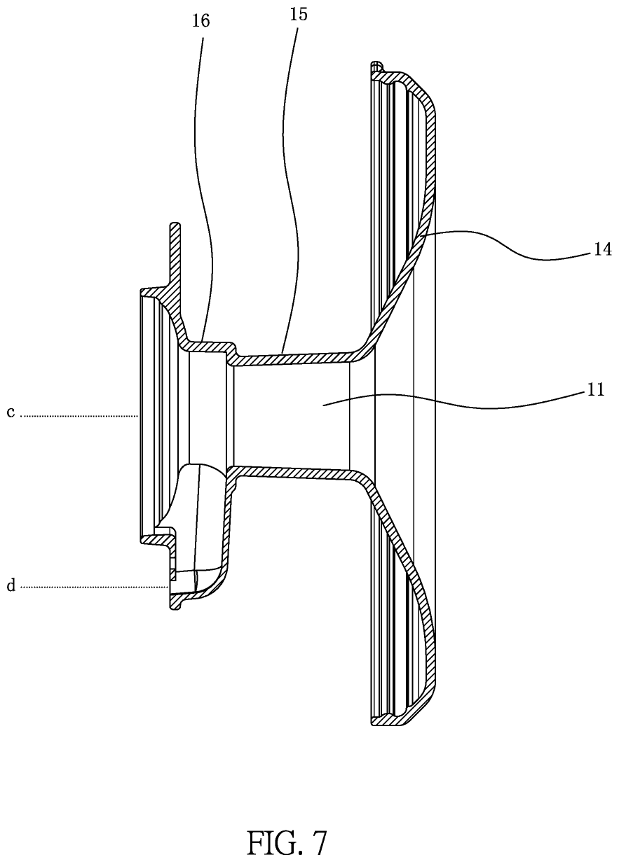

Furthermore, as shown in FIG. 7, the angle formed between the axis (c) of the membrane seat 16 and the normal line (d) of the plane formed by the opening 13 is set to be within the range of −5° to 5°, so as to match the structure of the membrane 3.

Further, refer to FIGS. 2 and 3, the fixing seat 24 is equipped with an annular groove 241 inside. The annular block 161 is positioned on the membrane seat 16, and it is surrounded outside the milk suction passage 11. The negative pressure diaphragm 31 is designed with a bending portion 34, and both sides of the bending portion 34 are in close contact with the walls of the annular block 161 and the annular groove 241, respectively. With the cooperation of the annular block 161 and the annular groove 241, the bending portion 34 of the negative pressure diaphragm 31 can be tightly pressed into the annular groove 241, ensuring excellent airtightness between the negative pressure diaphragm 31, the membrane seat 16, and the fixing seat 24.

Further, the bending portion 34 is equipped with a convex ring 36, which closely contacts the inner wall of the annular groove 241. The inclusion of the convex ring 36 further enhances the sealing performance between the negative pressure diaphragm 31 and the fixing seat 24, thereby preventing air leakage in the negative pressure cavity 4.

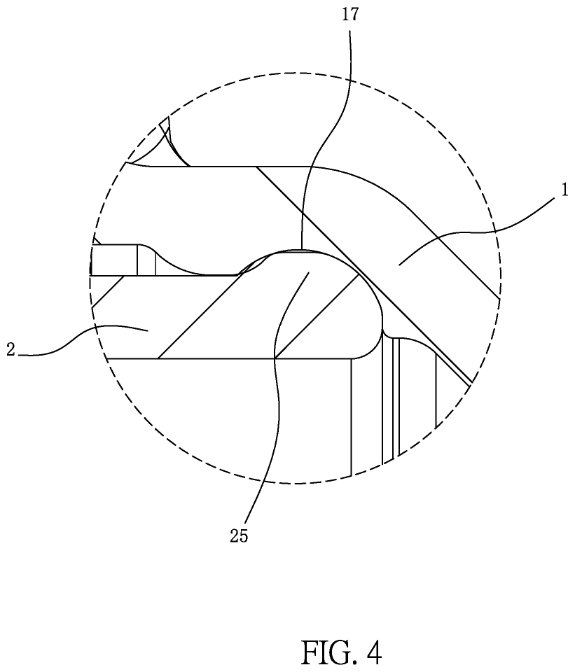

Refer to FIGS. 1 and 4, in the embodiment, the first shell 1 is equipped with an annular sealing groove 17, while the second shell 2 is fitted with an annular sealing ring 25 formed by protrusions, and the sealing ring 25 is positioned inside the sealing groove 17. By means of the interaction between the sealing ring 25 and the sealing groove 17, the first shell 1 and the second shell 2 are securely connected in a hermetic manner.

As a preferred solution in the embodiment, the sealing groove 17 is located on the cover part 14. The milk storage cavity 5 is formed by the walls of the cover part 14, the tube body 15, the membrane seat 16, and the second shell 2. When the device is used, milk can be stored in the milk storage cavity 5, and when a certain amount of milk has been collected, it can be poured out through the milk discharging hole 21. With this design, the breast milk collection device features a compact structure and is convenient for storage.

In this solution, together as shown in FIG. 2, both the first shell 1 and the second shell 2 are provided with a force-applying protrusion 18. The first shell 1 and the second shell 2 can be detached by applying opposing forces to the two force-applying protrusions 18.

In another preferred solution of this embodiment, the sealing groove is located on the membrane seat. The milk storage cavity is formed by the cooperation of the wall of the membrane seat and the wall of the second shell. The connecting part is provided on the second shell, and a connecting groove is disposed on an end portion of the connecting part, with an internal thread provided on the wall of the connecting groove. The milk discharging hole communicates with the connecting groove. The connecting part is used to connect a milk bottle, so when the milk in the milk accumulation chamber is discharged, it enters the milk storage cavity, flows out through the milk discharging hole 1, and is collected in the milk bottle.

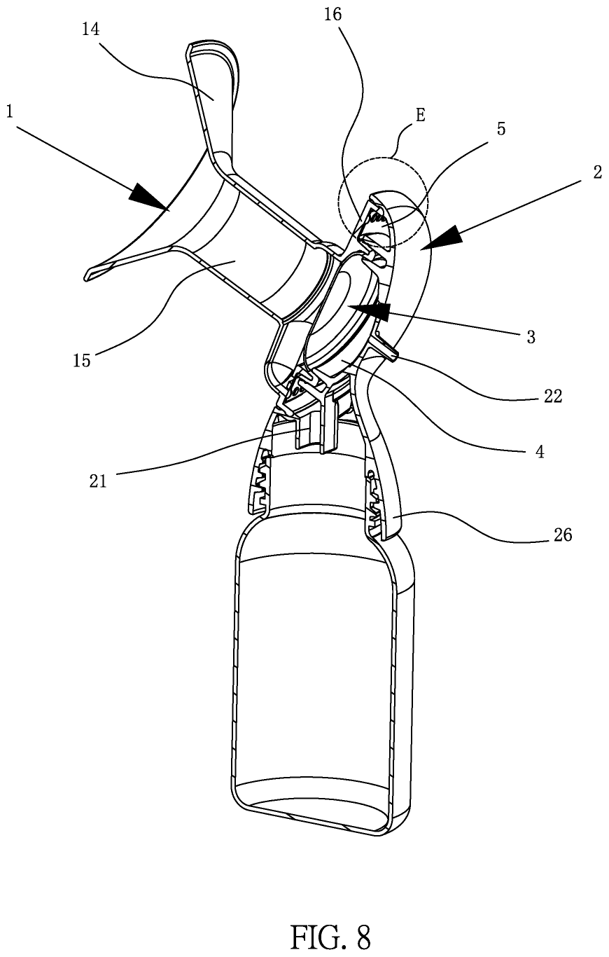

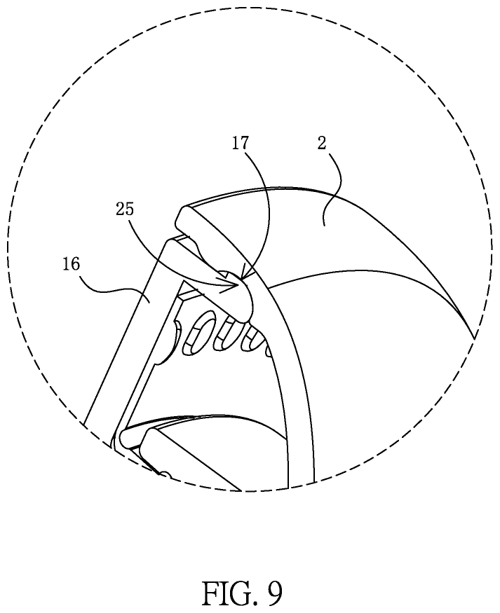

Next, refer to FIGS. 8 and 9, wherein FIG. 9 is an enlarged schematic diagram of the part E in FIG. 8, in another preferred solution of this embodiment, the sealing ring 25 is located on the membrane seat 16 and the sealing groove 17 is located on the second shell 2. The milk storage cavity 5 is formed by the cooperation of the wall of the membrane seat 16 and the wall of the second shell 2. The connecting part 26 is provided on the second shell 2, and a connecting groove is disposed on an end portion of the connecting part 26, with an internal thread provided on the wall of the connecting groove. The milk discharging hole 21 communicates with the connecting groove. The connecting part 26 is used to connect a milk bottle, so when the milk in the milk accumulation chamber 12 is discharged, it enters the milk storage cavity 5, flows out through the milk discharging hole 21, and is collected in the milk bottle. Compared with the previous embodiment, such a design allows the milk to enter the milk bottle directly without the need to first collect the milk in the milk storage cavity 5 and then pour it into other containers, making the collection of milk more convenient.

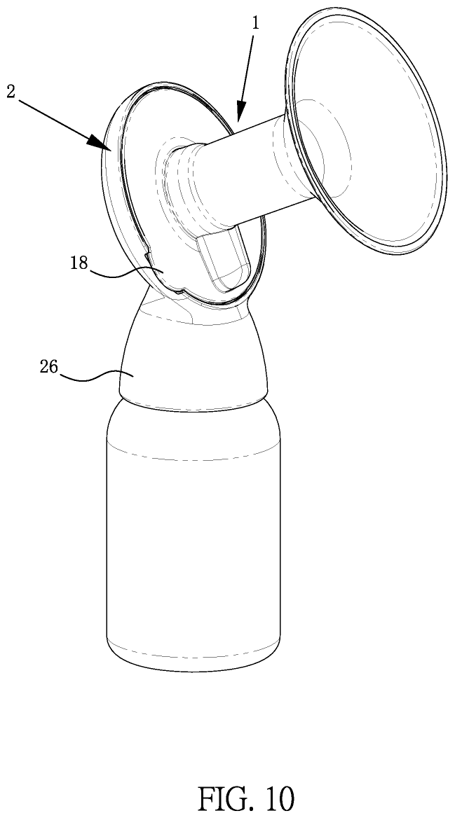

Refer to FIG. 10, which is a perspective view of FIG. 8 from another perspective, in this solution, only a force-applying protrusion 18 disposed on the first shell 1. The user can apply opposing forces to the force-applying protrusion 18 and the connecting part 26 to detach the first shell 1 from the second shell 2.

The disclosed breast milk collection device of the present invention addresses the problems of complex structure, inconvenient assembly, and short valve body lifespan in existing breast milk collection devices, which require frequent replacements and result in increased user consumption costs. This is achieved through rational structural design.

Even though numerous characteristics and advantages of certain inventive embodiments have been set out in the foregoing description, together with details of the structures and functions of the embodiments, the disclosure is illustrative only. Changes may be made in detail, especially in matters of arrangement of parts, within the principles of the present disclosure to the full extent indicated by the broad general meaning of the terms in which the appended claims are expressed.

What is claimed is:

1. A breast milk collection device, comprising:
a first shell, a second shell, and a membrane;
a middle portion of the first shell provided with a milk suction passage and a milk accumulation chamber outwardly formed by a side wall of the milk suction passage, and a side end of the milk accumulation chamber provided with an opening through the first shell;
a fixing seat disposed to the second shell, and the second shell hermetically connected to the first shell;
the membrane having a negative pressure diaphragm and an annular one-way valve diaphragm connected to an outer edge of the negative pressure diaphragm that the negative pressure diaphragm is installed on the fixing seat with an adjustable angle and closely contacts the first shell to block the milk suction passage;
a negative pressure cavity formed between the negative pressure diaphragm and the second shell;
the opening blocked by contact with the first shell by the one-way valve diaphragm;
a milk storage cavity formed by the cooperation of the first shell and the second shell on an outside of the fixing seat; and
a milk discharging hole and a negative pressure hole disposed on the second shell, wherein the milk discharging hole is connected to the milk storage cavity, and the negative pressure hole is connected to the negative pressure cavity.

2. The breast milk collection device of claim 1, wherein the second shell has a stopper protrusion on both sides of the opening, and the stopper protrusions are in contact with the one-way valve diaphragm.

3. The breast milk collection device of claim 1, further comprises a plurality of reinforcing protrusions disposed on the one-way valve diaphragm.

4. The breast milk collection device of claim 1, wherein the first shell has a cover part, a tube body, and a membrane seat, which are integrally formed; the cover part is trumpet-shaped; the milk suction passage is disposed from a middle portion of the cover part, and runs through the tube body and the membrane seat in turn; the milk accumulation chamber and the opening are disposed on the membrane seat; and the negative pressure diaphragm is in close contact with the membrane seat.

5. The breast milk collection device of claim 4, further comprising:
an annular groove disposed inside the fixing seat;
an annular block disposed on the membrane seat, and the annular block surrounded outside the milk suction passage; and
a bending portion disposed on the negative pressure diaphragm, and two sides of the bending portion are in close contact with a wall of the annular block and a wall of the annular groove respectively.

6. The breast milk collection device of claim 5, further comprising:
a convex ring disposed on the bending portion that the convex ring is in close contact with an inner wall of the annular groove.

7. The breast milk collection device of claim 4, further comprising:
an annular sealing groove formed on the second shell; and
an annular sealing ring formed by protrusions on the first shell, wherein the sealing ring is located inside the sealing groove.

8. The breast milk collection device of claim 1, further comprising:
a connecting part disposed on the second shell, wherein an end of the connecting part is provided with a connecting groove communicated with the milk discharging hole, and a wall surface of the connecting groove is provided with an internal thread.

9. The breast milk collection device of claim 1, further comprising:
an annular sealing groove formed on the first shell; and
an annular sealing ring formed by protrusions on the second shell, wherein the sealing ring is located inside the sealing groove.

10. The breast milk collection device of claim 1, wherein an included angle formed between an axis of the negative pressure diaphragm and a normal line of the one-way valve diaphragm is within the range of −5° to 5°.

11. The breast milk collection device of claim 4, wherein an included angle formed between an axis of the membrane seat and a normal line of the plane where the opening is located is within the range of −5° to 5°.
